(19) United States
(12) Patent Application Publication
(10) Pub. No.: US 2024/0321406 A1
(43) Pub. Date: Sep. 26, 2024

(54) PREDICTION OF PHARMACOKINETIC CURVES

(71) Applicant: Hoffmann-La Roche Inc., Little Falls, NJ (US)

(72) Inventors: Dominic BRÄM, Basel (CH); Neil PARROTT, Basel (CH); Lucy HUTCHINSON, Basel (CH); Bernhard STEIERT, Basel (CH)

(21) Appl. No.: 18/579,293

(22) PCT Filed: Jul. 14, 2022
(86) PCT No.: PCT/EP2022/069799

§ 371 Date: Jan. 12, 2024

(30) Foreign Application Priority Data
Jul. 15, 2021 (EP) 21185957.4

(51) Int. Cl. G16C 20/30; G16C 20/70

(52) U.S. Cl. CPC G16C 20/30; G16C 20/70

(57) ABSTRACT

A computer-implemented method of predicting at least one future point on a pharmacokinetic curve for a given species comprises: receiving an input comprising data representing a sequence of concentration-time points of a pharmacokinetic curve, each concentration-time point indicative of an amount of the given species in a subject's body at a respective time; applying a machine learning model to the input data, the machine learning model configured to generate an output comprising at least one subsequent concentration-time point in the pharmacokinetic curve. Computer-implemented methods of training machine learning models are also provided.

### TECHNICAL FIELD OF THE INVENTION

The present invention relates to computer-implemented methods of predicting at least one future point on a pharmacokinetic curve for a given species, the methods making use of a machine learning model. Other aspects of the invention relate to generating the machine learning models in question.

### BACKGROUND TO THE INVENTION

Pharmacokinetics describes the field of quantitative and qualitative analysis of pharmacological data through modelling, e.g. the modelling of pharmacokinetic (PK) data from a clinical or preclinical study¹. It is an integral part of the approval of new pharmaceutical products through health authorities and a key element for personalized dosing. Even though the state-of-the-art methods in pharmacometrics have proven their usefulness over many years, they still have some drawbacks in terms of efficiency and their requirement for a high level of expertise of the modeller. We would like to propose a new approach based on advanced computational methods, which could supplement the state-of-the-art methods and help to increase efficiency in pharmacometrics.

One state-of-the-art approach in pharmacometrics is population PK modelling² where, drug concentration data is described through a structural model with well-defined equations including parameters related to physiological processes. This model is then typically fitted to clinical data to estimate population values and individual values for these parameters. In a second step, quantitative relationships between the individual parameters and patients' characteristics, i.e. covariates, are investigated.

The estimation of population and individual values for the model parameters is typically based on mixed effects modelling³. Several software tools (e.g. NONMEM, Monolix, R) with different mathematical estimation algorithms (e.g. FOCEI, SAEM) are available for this task. Depending on the size of the data set, the software used, the estimation algorithm and the complexity of the structural model, one run of parameter estimation may take from a few minutes up to several hours. In the model building process, where the final structural model is developed by iteratively fitting a candidate structural model, assessing its goodness-of-fit, identifying possible systematic residual errors and adjusting the structural model accordingly, multiple runs must usually be performed⁴. In addition to the model building process, the process of covariate selection also requires multiple rounds of parameter calibration, especially when the stepwise covariate modelling approach is used. Considering the model building process and covariate selection, a large number of runs must usually be performed to obtain an appropriate PK model to describe the data and therefore the entire process can take at least several days. Additionally, the resulting model is highly dependent on the expertise of the pharmacometrician who performs the PK modelling.

The present invention aims to address these drawbacks, by providing a reliable method of predicting pharmacokinetics in a manner which is efficient and no longer dependent on the expertise of the pharmacometrician performing the modelling.

### SUMMARY OF THE INVENTION

The present invention provides a new approach to making pharmacokinetic predictions, which takes advantage of recent advances in computational methods which are less time-consuming. In broad terms, the present invention addresses the problems with conventional, or “traditional” pharmacokinetic modelling by applying machine learning techniques. Specifically, a machine learning model such as an artificial neural network (ANN) 5 is used to determine future points on a pharmacokinetic curve, rather than a pharmacokinetic model. As we will explain in this application, machine learning methods are applicable to the prediction of the next points on a PK curve both in the presence and absence of dosing. The method may also be modified to take into account the PK profiles of different drugs, and different patients.

A first aspect of the present invention provides a computer-implemented method of predicting at least one future point on a pharmacokinetic curve for a given species, the computer-implemented method including: receiving an input comprising data representing a sequence of concentration-time points of a pharmacokinetic curve, each concentration-time point indicative of an amount of the given species in a subject's body and/or tissue at a respective time; applying a machine learning model to the input data, the machine learning model configured to generate an output comprising at least one subsequent concentration-time point in the pharmacokinetic curve. Optionally, the computer-implemented method may further comprise receiving the output from the machine learning model. Concentration-time points are referenced above. This should be interpreted broadly as covering e.g. concentration measured in absolute or relative terms, using any standard units of concentration. Concentration data may be normalized by e.g. dividing each concentration value by a maximum initial concentration value, or a maximum concentration value following administration of a dose.

Within the context of the present invention, a pharmacokinetic curve is a curve which indicates the change in the amount of a given species in a user's body over time. Herein, the term “species” is intended to refer to a specific compound, substance, or chemical present in a user's body. Preferably, the species in question is a pharmaceutical compound such as a drug or medicine, or any other pharmacological compound, including its downstream products following biotransformation or metabolism, i.e. metabolites or any processed version of the pharmacological compound. Herein “the amount of a given species in a user's body” refers to a measure of how much of the given species is present in tissue of a user, or in a bodily fluid of the user. The bodily fluid may include a sample of tissue/organ of a subject, and/or of a product produced by a tissue/organ of a subject. A product produced by a tissue/organ of a subject may e.g. be a product of secretion (e.g. a glandular secretion, milk, colostrum, tears, saliva, sweat, cerumen, mucus), sputum, semen, vaginal/cervical fluid, blood (plasma, serum), cerebrospinal fluid (CSF), a product of excretion, faeces, or urine, skin or hair. In most cases, the bodily fluid will be blood, particularly plasma, but it should be noted that the present invention would remain effective for all other bodily fluids. The above list is not exhaustive.

Before the step of receiving the input comprising the data representing a sequence of concentration-time points of a pharmacokinetic curve, the method may comprise a step of measuring, at a plurality of time points, the concentration of the species in the body of the subject, and generating the input, which is then subsequently received in the computer-implemented method of the first aspect of the invention. The measurement may take place using any known methods. Even if the method does not explicitly include the measurement step, the received input may comprise data obtained from measurements of the concentration of the species in the body of the subject at a plurality of time points. Using “real” data obtained from a subject may be advantageous because all subjects respond differently to drugs or other species. So, by using patient data, and feeding these into the machine learning model, it is possible to obtain concentration-time predictions which are specific to that individual. It can thus be possible to select a dosing regimen for the specific patient (more detail later in this application).

After receiving the output from the machine learning model, the output including at least one subsequent concentration-time point in the pharmacokinetic curve, the computer-implemented method may further include a step of generating instructions, which when received by e.g. a computer (or a processor or display thereof), cause a display component of the computer to display the pharmacokinetic curve including at least the sequence of concentration-time points forming the input, and the output concentration-time point. Alternatively, put the computer-implemented method may further include a step of displaying the pharmacokinetic curve including at least the sequence of concentration-time points forming the input, and the output concentration-time point.

Preferably, the subsequent concentration-time point of the output is the next concentration-time point after the sequence of concentration-time points forming the input. It is preferable that the sequence concentration-time points forming the input are evenly spaced in time, i.e. each consecutive pair of points is separated, temporally, by the same interval. Then, the time coordinate of the output subsequent concentration-time point is preferably spaced from the time coordinate of the final point in the input sequence of concentration-time points by the same interval.

Embodiments of the first aspect of the present invention may be used to predict at least one subsequent concentration-time point in a pharmacokinetic curve when no dose of the given species has been administered, or to predict at least one subsequent concentration-time point in the pharmacokinetic curve after a dosing event. In some embodiments, the computer-implemented method of the first aspect of the invention may be used to predict both of the above.

In some cases, the machine learning model may be configured to generate an output comprising a plurality of subsequent concentration-time points on the pharmacokinetic curve. For example, the machine learning model may be configured to generate a first output concentration-time point, and the computer-implemented method may include an additional step of applying the machine learning model to a sequence of concentration-time points including the first output concentration-time point, to generate a second output concentration-time point. The process may be repeated until a desired number of output concentration-time points have been generated. The number of concentration-time points forming the input may be the same each time, or it may include the whole original input sequence, and the subsequently-generated output concentration-time points.

In preferred cases, a separate machine learning model may be adapted for each the two functions. Throughout this application, these will be referred to as the curve model and the dose model. We begin with a discussion of the general features of the machine learning models which may be employed in embodiments of the computer-implemented method of the first aspect of the invention. We then discuss some specific details of the curve model, then the dose model. After that, we discuss the cases in which both models are employed.

The curve model is a machine learning model. In the context of the present application, the term “machine learning model” is used to refer to a machine learning algorithm which has been trained. It could be said that the machine learning algorithm is a combination of the machine learning algorithm and the training data. We discuss the training of the machine learning model later in the application. In preferred cases, the machine learning model of the present invention comprises an artificial neural network (ANN).

Artificial neural networks (here, just “neural networks”) belong to the family of supervised machine learning models, and are able to approximate linear and nonlinear functions by creating a network of calculations steps and calibrating the model parameters of this network to the training data⁶. In this application, the model parameters will be referred to as “weights” or “biases”, and the calibration of these weights/biases is referred to as “training” the neural network. The architecture of a neural network is structured in three sections: an input layer, one or more hidden layers, and an output layer. The input layer defines the information that is provided to the network, and may be referred to as the independent variables, in mathematical terminology. In the present case, the input layer receives the data representing the sequence of concentration-time points. In some cases (discussed in more detail later), the input may further comprise data representing e.g. patient characteristics and/or information regarding the given species. The output layer represents the dependent variables, i.e. a predicted outcome, in this case at least the subsequent concentration-time point. The hidden layers define the calculation steps which lead from the input layer to the output layer. For different calculations and data types, different types of hidden layer may be used.

Specifically, the neural network may comprise one or more long short-term memory (LSTM) layers. Nodes in an LSTM layer differ from e.g. standard feedforward neural network nodes in that an LSTM layer includes feedback connections as well as feedforward connections. LSTM layers are particularly well adapted for handling temporal sequences of data points, such as the sequences of concentration-time points which form (at least part of) the input in the present case. A detailed explanation of LSTM layers may be found in Hochreiter et al. (1997)⁷.

The ANN may further include one or more densely connected layers. A densely connected layer in a neural network is a layer in which each node is connected to every node in the previous layer of the neural network. Densely connect layers, or dense layers, are used to handle static data points. The means by which each type of layer may be used will be explained in more detail later on.

Simple neural networks, e.g. with one hidden layer and a few weights may be expressed as an explicit function (which is equivalent to nonlinear regression), the strength of neural networks lies in the possibility to largely increase their complexity by increasing the number of hidden layers. In this way, neural networks can approximate highly complex functions. It should be stressed that the weights which represent the parameters of the neural network typically do not represent physiologically meaningful parameters. Rather, they are comparable to parameters from a regression model.

The neural network is preferably a trained neural network. In the context of the present invention, training is the process by which the weights of the neural network are calibrated, as described in Kavzoglu et al. (1999)⁸. A neural network is trained using training data, which includes output data for each input data. In order to make useful predictions, the training data should be representative for the setting in which the neural network is to be applied. The weights of the neural network are adjusted during training, usually using a gradient based method, in order to minimize a loss or objective function assessing the difference between the predicted and observed results. With an increasing number of hidden layers and weights to calibrate, an increased training data set is required to manage the risk of overfitting. If, for a specific problem, no sufficiently large data set is available, the concept of transfer learning can be used, as described in Weiss et al. (2016)⁹. A neural network previously trained on a similar, but not the same, problem can be retrained on a new data set. Since the previously trained neural network has already learned to accomplish a particular task, some of the weights need not be adjusted and can be fixed for the retraining. The lower number of adjustable weights means that the training data set can be smaller, for the retraining than it was for the original training. Transfer learning proves particularly useful when modifying trained machine learning models to take account of e.g. patient profiles or species profiles, as discussed later in this application.

We now turn to some of the specifics of the curve model. The curve model is a machine learning model, and may comprise a curve network, which is an artificial neural network having the properties set out above. Specifically, when the curve network is applied to the input sequence, it is configured to output one or more subsequent concentration-time points which would be expected in the absence of any recent dosage of the given species. In other words, the curve network is configured to predict the concentration-time points of the given species, preferably in the elimination phase, in the absence of dosing events or any other stimuli. As discussed previously, the curve network may be configured to predict a plurality of subsequent concentration-time points. This may be done iteratively, i.e. after the curve network has output a first subsequent concentration-time point, the computer-implemented method may further comprise applying the curve network again to the combination or concatenation of the initial input sequence of concentration-time points and the first subsequent concentration-time point. This may be repeated as required in order to predict a plurality of concentration-time points on the pharmacokinetic curve. Specifically, the computer-implemented method may comprise: applying the curve network to the input data to generate an output comprising a first subsequent concentration-time point; generating new input data, wherein the new input data comprises at least the original input data and the first subsequent concentration-time point; and applying the curve network to the new input data to generate an output comprising a second subsequent concentration-time point. This may be generalized to a general plurality of concentration-time points as following. The computer-implemented method of the first aspect of the invention may comprise: (a) applying the curve network to the initial input data to generate a subsequent concentration-time point; (b) applying the curve network to updated input data, the updated input data comprising the initial input data and all subsequently-generated concentration-time points; and (c) repeating step (b). Alternatively, rather than including all of the initial input data, the updated input data may include the same number of concentration-time points on each iteration, i.e. for every new subsequent concentration-time point which is added, the earliest concentration-time point from the initial input data is removed.

In a preferred case, the curve network may comprise at least one LSTM layer which is configured to decompose the sequence of concentration-time points forming the input into parameters representative of the sequence. In a preferred case, the curve network includes a first LSTM layer and a second LSTM layer which are configured to perform this task. With each subsequent LSTM layer, the abstraction level of the curve increases, and the data are smoothed. It has been observed by the inventors that the use of two abstraction levels worked particularly well. In addition to the at least one LSTM layer, the curve network preferably comprises a densely connected layer which is configured to process the parameters in order to predict the at least one subsequent concentration-time point. Preferably, the densely connected layer is configured to combine the parameters in a nonlinear manner. In order to provide an effective machine learning model, the curve network must be trained. The manner in which the neural network is trained is discussed later in this application, with reference to the second aspect of the invention. For now, it suffices to say that the machine learning model, specifically the curve network, is preferably trained using the computer-implemented method of the second aspect of the invention.

We now discuss the dose model. The dose model is a machine learning model, and may comprise a dose network, which is an artificial neural network having the properties set out earlier in this application. In addition to the sequence of concentration-time points, the input data for the dose network preferably includes dosage data. The dosage data may include at least one value of a dosage to be administered, and optionally information representing a time or plurality of times at which the dosage (or dosages) is to be administered. Alternatively, the dosage data may comprise an input sequence of concentration-time points which cover a measured or simulated initial dosing event. Then, the dosage data may further comprise information indicating the time or times at which a subsequent dose or doses are to be received, and the size of those doses relative to the initial dose. It will be appreciated that in this case, no absolute dosage data is required, since the patient's response to the first dosing event takes this into account already.

The sequence of concentration-time points are preferably provided in the form [c₁, c₂, c₃, . . . , cₙ] where cᵢ is the concentration at time tᵢ. In such cases, the dosage data may be provided in the form of a sequence [d₁, d₂, d₃, . . . , dₙ], where dᵢ is the concentration at time tᵢ. Since the dose is only likely to be occasionally administered, the majority of the values dᵢ will be zero. In some cases, the values dᵢ will be relative values; specifically the dosage data may include an absolute value of an initial dosage, and dosage data in the form [d₁, d₂, d₃, . . . , dₙ], where dᵢ represents the size of each dose relative to the initial dosage (i.e. dᵢ=1, and d₂ onwards represent the ratio of that dosage to dᵢ) at time tᵢ.

More details of the dosage data will be described later in this application.

The purpose of the dose network is to predict an increase, if any, in concentration of the given species after the administration of a dose of the given species. In other words, the dose network is configured to output one or more values indicative of an increase in concentration of the given species after a dose has been administered (the dose being the amount specified in the dosage data), preferably in the form of concentration-time points. In some implementations, the dose network predicts only the first concentration-time point after a given subsequent dose has been administered. In other cases, the dose network may be used to predict a plurality of concentration-time points after the administration of the dosage. For example, in addition to being trained to predict the increase in concentration, the dose network may further be trained to predict the shape of the pharmacokinetic curve immediately after the dosage is administered. This may take place using an analogous iterative procedure as was discussed in respect of the curve network, or it may simply be based on the input sequence covering the first dosing event.

We now discuss the structure of the dose network, as we did for the curve network. The dose network may comprise two sub-networks: a sequence sub-network and a dosage sub-network, wherein the sequence sub-network is configured to receive and process the portion of the input data comprising the sequence of concentration-time points, and the dosage sub-network is configured to receive and process the portion of the input data comprising the dosage data. Each of the sub-networks may comprise at least one LSTM layer, preferably a first LSTM layer and a second LSTM layer configured to decompose their respective inputs into parameters representative of the information contained in the respective inputs (i.e. the sequence data and the dosage data). Following the LSTM layers, each sub-network preferably comprises at least one densely connected layer configured to combine the parameters in a nonlinear fashion, as was the case for the curve network. In a preferred implementation there are three densely connected layers in each sub-network. Each sub-network, after processing using the various LSTM and/or densely connected layers generates an output, which may be in the form of a vector. At this stage, the respective outputs of each sub-network are unlikely to correspond to any meaningful physiological parameters.

Therefore, in order to derive a meaningful result, the dose network preferably includes a further combination sub-network, which is configured to combine the outputs from the dosage sub-network and the sequence sub-network. In some cases, combining the outputs comprises: concatenating the outputs (e.g. concatenating the vectors) and processing the resulting concatenation using at least one densely connected layer.

Preferably, as before the sub-network comprises three densely connected layers. The combination sub-network may comprise a concatenation module which is configured to perform the concatenation step. The output of the combination sub-network is a parameter indicative of the increase in concentration of the given species as a result of the administration of a dosage or plurality of doses as described by the dosage data, preferably in the form of a concentration-time point.

In simple cases, the dosage data may simply be in the form of information about a single dose of the given species. In this case, the resulting pharmacokinetic curve will include a peak a short time after the dose is administered, followed by a gradual decay as the drug is metabolized or otherwise cleared by the body. In order to predict the resulting pharmacokinetic curve, the curve model and the dose model may be used in parallel with each other: the dose model predicts the concentration after the initial dosage (which effectively represents the response of the body to the initial dose), and the curve model predicts preferably the subsequent decay in concentration. In order to get a picture of the combined effect of the initial increase in concentration as a result of the dosage, as well as the underlying decay in concentration, the computer-implemented method preferably includes a step of combining the output of the dose model with the output of the curve model. In preferred cases, combining simply comprises adding the outputs together.

Often, it is desirable to predict the pharmacokinetic curve resulting from a dose regimen, i.e. a series of doses at predetermined intervals. In these cases, the pharmacokinetic curve (i.e. the concentration-time profile) includes a series of peaks or spikes, with a decay phase after each. The concentration then peaks again with the administration of the subsequent dose. Embodiments of the computer-implemented method of the present invention are able effectively to predict such a pharmacokinetic curve using a combination of the processes which have been outlined already. In order to handle a dose regimen in this manner, the dosage data preferably includes at least the following: the absolute amount of the initial dosing event or an input sequence which includes the pharmacokinetic response to a first dosing event; and the times and values of one or more subsequent dosing events. The values of the subsequent dosing events may be provided in absolute terms, or preferably in relative terms (i.e. a ratio of the subsequent dosages to the first dosage). This data is input into the dosage sub-network, as before. The input data to the sequence sub-network in these cases preferably includes a sequence of concentration-time points either measured or predicted from the first dosing event. The number of concentration-time points in the input data to the sequence sub-network is preferably selected so that the peak concentration value rests within that sequence of concentration-time points. Using this information, the dose network is then able to predict the resulting increase in concentration as a result of each of the dosing events described in the dosage data. As discussed, the dose network may predict only the initial increase, or it may predict a plurality of concentration-time points. The curve network operates in the usual manner, predicting the preferably continual decay of the concentration. Then, as before the outputs of the curve model and the dose model are combined, preferably by addition, to generate the overall output in the form of a series of concentration-time points. The iterative process described previously is preferably used to generate the series of concentration-time points. It will be appreciated that for the concentration-time points which fall between dosing events, there is no contribution to the overall output from the dose model, since it is only used to predict the short-term system response to a dosing event, for example the dose network output may comprise concentration-time points for a period of within about 5 to 15 hours of the administration of a dose, more preferably within about 6 to 14 hours of the administration of a dose, more preferably within about 7 to 13 hours of the administration of a dose, more preferably within about 8 to 12 hours of the administration of a dose, more preferably within about 9 to 11 hours of the administration of a dose, and most preferably within about 10 hours of the administration of a dose. The amount of time after the dose depends on e.g. the nature of the given species, and/or the method of administration of the dose.

The output of the computer-implemented method of the first aspect of the invention comprises at least one subsequent concentration-time point in a pharmacokinetic curve. In some cases, the output may comprise a plurality of concentration-time points. Before discussing a second aspect of the present invention, we consider how the data forming the output of the machine learning model may be used in a clinical setting. The computer-implemented method may be particularly useful for determining appropriate dosing regimens, either in a general context or in the context of personalized dosing. Broadly speaking, determination of a dosing regimen may be achieved by running a simulation several times, each time corresponding to a different dosing regimen, to generate several respective outputs. Then, the computer-implemented method may comprise selecting an input corresponding to a particular output. The selection may be based on various parameters which may be extracted from the results.

More specifically, the computer-implemented method may further comprise a step of determining a dosing regimen based on the generated output comprising at least one subsequent concentration-time point. We have explained above that the output may comprise a plurality of future concentration-time points. Herein, we refer to the plurality of future concentration-time points as a future concentration-time curve or a future pharmacokinetic curve. The computer-implemented method may comprise receiving a plurality of inputs, thereby generating a respective plurality of outputs. Each input and its corresponding output (e.g. future concentration-curve) may correspond to a respective dosing regimen. Then, determining a dosing regimen may comprise selecting a dosing regimen corresponding to one of the inputs based on its respective output.

We now discuss the nature of the inputs in a little more detail. Each input preferably comprises a sequence of concentration-time points representing a dosing regimen. The plurality of inputs may vary in one or more of: the dosing level (i.e. the concentrations as a result of the dosing regimen) and the frequency of the doses.

Determination of the dosing regimen based on the generated future concentration-time curve may comprise, for each output, determining the values of one or more pharmacological parameter, and selecting the output based on the value of the pharmacological parameter. The pharmacological parameter may comprise the area under the determined future concentration-time curve or AUC (this is not to be confused with an AUC which is used to determine the classification accuracy of a machine-learning model). The AUC is indicative of the exposure of a subject to a particular species or drug. In pharmacology and pharmacokinetics, the term “exposure” is used to refer to the amount of a drug or other species which a patient is exposed to over time. It is important that the exposure is no less than an efficacy threshold at which the drug or other species has a useful clinical effect, and no more than a toxicity threshold, at which the drug or other species risks harming the subject. These thresholds correspond to respective AUC thresholds. Another pharmacological parameter which may be used is the maximum concentration. It is also preferable that the maximum concentration is no less than an efficacy threshold and/or no more than a toxicity threshold. Another pharmacological parameter is the minimum (or “trough”) concentration. For some cases, it is preferable that the minimum concentration is no less than an efficacy threshold and/or that the minimum concentration is no more than a toxicity threshold. In some cases, it is preferred that the minimum concentration is no less than an efficacy threshold and the maximum concentration is no more than a toxicity threshold.

Accordingly, determination of the dosing regimen may comprise selecting one or more dosing regimens corresponding to an input, the AUC calculated for the output corresponding to which is no less than an efficacy AUC threshold, and/or no more than a toxicity AUC threshold. Alternatively, or additionally, determination of the dosing regimen may comprise selecting one or more dosing regimens corresponding to an input, the maximum concentration derived from the output corresponding to which is no less than an efficacy maximum concentration threshold, and/or no more than a toxicity maximum concentration threshold. Alternatively, or additionally, determination of the dosing regimen may comprise selecting one or more dosing regimens corresponding to an input, the minimum concentration derived from the output corresponding to which is no less than an efficacy minimum concentration threshold, and/or no more than a toxicity minimum concentration threshold. Alternatively, or additionally, determination of the dosing regimen may comprise selecting one or more dosing regimens corresponding to an input, the minimum concentration derived from the output corresponding to which is no less than an efficacy minimum concentration threshold, and the maximum concentration derived from the output corresponding to which is no more than a toxicity maximum concentration threshold.

For some drugs or other species, there is a minimum inhibitory concentration (MIC), which is the minimum concentration of the drug or other species required for it to have a therapeutic effect. This is particularly true for some antibiotics. In order to provide a consistent effect, it is preferred that the concentration of the drug or other species exceeds the MIC for a certain proportion of time, e.g. between doses. Thus, the pharmacological parameter may comprise the proportion of time for which the concentration of the drug or other species exceeds the MIC, referred to herein as t_{MIC}. Accordingly, determination of the dosing regimen may comprise selecting one or more dosing regiments for which t_{MIC} is no less than an efficacy threshold value. This ensures that there is sufficient drug or other species in the user's body to ensure a consistent therapeutic effect throughout the course of treatment.

The ratio of AUC and MIC (i.e. AUC/MIC) is also an important pharmacological parameter, and may be subject to the same thresholds as the AUC.

The thresholds may be predetermined, or they may be determined in an additional determination step.

In addition to determination of a generalized dosing regimen for a particular drug or other species, computer-implemented methods according to the present invention may be used to develop a personalized dosing regimen. Personalized medicine is a rapidly advancing field in which rather than prescribing standard treatments at standard doses, the treatment plan is hand-picked for a patient. This is often done ad hoc by clinicians, but the present invention allows a more systematic approach by predicting the pharmacokinetic response to various dosing regiments, and indeed different drugs or other species. The personalized dosing approach may be very similar to the approach for determination of more generalized dosing regimens, and will not be repeated here. A difference is that the inputs may vary not only in the dosing level or the frequency of the doses, but also the drug or other species itself. Another difference may be that the efficacy and/or toxicity thresholds may be calculated specifically for the subject in question, e.g. based on existing historical or physiological data. In this way, a subject's responses to different dosing regimens of different drugs or other species can be systematically assessed in order to determine an appropriate course of treatment.

The preceding disclosure focuses on the computer-implemented method for actually predicting future concentration-time points in a pharmacokinetic curve. The computer-implemented method relies heavily on the action of a machine learning model, which itself may comprise a dose model and/or a curve model, each with separate functions, and which may be combined to produce an overall output. It will be appreciated that a machine learning model must typically be trained before it can be effective, and that training a machine learning model requires training data. Accordingly, a second aspect of the present invention provides a computer-implemented method of generating a machine learning model for predicting at least one future point on a pharmacokinetic curve for a given species, the computer-implemented method comprising: providing a machine learning algorithm; receiving training data, the training data comprising a plurality of pharmacokinetic curves; and training the machine learning algorithm using the received training data, thereby generating the machine learning model.

As has been explained previously, the machine learning model may comprise two neural networks: a curve network and a dose network. The function of each of these networks is different, so in preferred cases, different training data may be used for each network. A given pharmacokinetic may be divided into two types of region: dose-effect regions and curve-effect regions. Dose-effect regions of the pharmacokinetic curve are those regions occurring at the time of, and for a predetermined amount of time (or number of concentration-time points) after the administration of a dose. The concentration of the given species in the dose-effect region (s) are assumed still to be governed or influenced by the administration of the dose. In some cases, the dose-effect regions may comprise the regions of the pharmacokinetic curve within about 5 to 15 hours of the administration of a dose, more preferably within about 6 to 14 hours of the administration of a dose, more preferably within about 7 to 13 hours of the administration of a dose, more preferably within about 8 to 12 hours of the administration of a dose, more preferably within about 9 to 11 hours of the administration of a dose, and most preferably within about 10 hours of the administration of a dose. The curve-effect regions are the regions outside of the dose-effect regions, where it is assumed that the concentration of the given species is no longer governed or influenced by the administration of the dose. In the context of this application, the dose network may be used to predict concentration-time points in the dose-effect region (s) only, whereas the curve network may be used to predict concentration-time points in both the dose-effect regions and the curve-effect regions.

Training the machine learning algorithm is preferably done by supervised learning. Specifically, the training data preferably includes associated pairs of training data items, the pairs each including an input sequence of concentration-time points, and at least one output concentration-time point, which is preferably the point in the concentration-time curve which follows the final point in the input sequence. In some cases, there may be a plurality of output concentration-time points, for example in order to train algorithms whose function is to predict a plurality of subsequent concentration-time points.

For the curve network, the pharmacokinetic curves included in the training data may include single dose curves and/or multiple dose curves. In some cases, the pharmacokinetic curves may each be split into a plurality of smaller concentration-time profiles, the smaller concentration-time profiles forming at least part of the training data. To train the curve network, preferably only portions of the pharmacokinetic curves in the curve-effect regions are used, in order to ensure that the curve network is trained only on data representing the decay in the concentration of the given species, without any influence from the dose. The input sequence of concentration-time points preferably includes at least 5 points, more preferably at least 6 points, more preferably at least 7 points, more preferably at least 8 points, and most preferably at least 9 concentration-time points. Training the curve network preferably comprises using an Adam¹⁰ optimizer for parameter optimization, and a mean-squared error as a loss function.

In order to train the weights of the dose network, the pharmacokinetic curves included in the training data may include single dose curves and/or multiple dose curves. In some cases, the pharmacokinetic curves may be split into a plurality of smaller concentration-time profiles, the smaller concentration-time profiles forming at least part of the training data. To train the dose network, preferably only portions of the pharmacokinetic curves in the dose-effect regions are used, in order to ensure that the dose network is trained only on data representing the increase in concentration of the given species as a result of the administration of the dose. As discussed previously, the dose network preferably takes two inputs: an input sequence of concentration-time points and dosage data. These data preferably form the input when training the dose network. Then, the output data may include at least one output concentration-time point, and in some cases, a plurality of output concentration-time points. The rationale for the output data including a plurality of concentration-time points is that the effect of the administration of the dose may be seen for points other than the first point which occurs immediately after the administration of the dose. For example, in the case of oral administration of a drug, the effects of the actual administration of the dose may be seen around 10 hours after the administration of the dose, e.g. as the drug is metabolized and absorbed into the bloodstream via the digestive system. Appropriate ranges for the amount of time during which the dose effect should be considered have been set out elsewhere in the application.

In one example, in which the machine learning algorithm includes a dose network and a curve network, training the machine learning algorithm may include the following steps: training the curve network using curve network training data, thereby establishing a plurality of curve network weights; fixing the curve network weights; inputting the training data, wherein the training data comprises at least an input sequence of concentration-time points including a peak concentration-time point immediately after the administration of a dose, and dosage data; inputting the output data, wherein the output data comprises at least one concentration-time point, preferably the point immediately following the final point in the input sequence of concentration-time points, as would be determined by the whole machine learning algorithm. In order to determine the weights of the dose network, the whole machine learning algorithm is then trained using this data. In other words, the output data represents the output not only the data which would be predicted using the dose network alone, but the data which would be predicted using the whole machine learning algorithm (e.g. by combining the input sequence and dosage data, to generate a value for the increase in concentration, and then to add this to the general trend identified by the curve network). It is beneficial to hold the weights of the curve network fixed because the weights parameterize the curve-effects only, and are independent from the dose-effects. By holding the curve network weights, the whole training process is also more computationally efficient.

As with the curve network, an Adam optimizer and mean-squared error loss function may still be used.

In some cases, the training data may be real data, as measured from real patients or subject's, or obtained from clinical studies. Alternatively, the training data may be simulated training data. The training data may be generated using known pharmacokinetic models, preferably physiologically-based pharmacokinetic models. It should also be noted that simulated data can also be used to test the machine learning model.

The concept of transfer learning has been discussed already in this application. It is particularly applicable when it comes to training neural networks. In the context of the present invention, transfer learning is very useful for retraining the machine learning model to work for patients whose pharmacokinetic characteristics are different, e.g. due to a medical condition or disease. Specifically, a patient or medical condition may be associated with a respective patient profile or medical condition profile which defines the effects of a medical condition on a patient's pharmacokinetic characteristics. For brevity, we refer simply to the “profile” in the subsequent description. In preferred cases, the profile may include a plurality of parameters of a pharmacokinetic model which may be used to simulate the pharmacokinetic behaviour of a particular patient, or a general patient having the medical condition. Preferably, the pharmacokinetic model is a physiologically-based pharmacokinetic model. Retraining the model may comprise retraining the machine learning algorithm using new training data, the new training data based on a pharmacokinetic model such as a physiologically-based pharmacokinetic model, and the profile. Specifically, the computer-implemented method may include a step of generating simulated pharmacokinetic curve data using the physiologically-based pharmacokinetic model and the data in the profile, and retraining the data based on the simulated data. In some cases, transfer learning may be employed in order to improve the efficiency of the training process. Specifically, when retraining the machine learning model, some of the weights in some of the layers may be held constant. For example, the weights in any or all of the LSTM layers of the curve network, the densely connected layers of the curve network, the LSTM layers of the dose network, and the densely connected layers of the dose network may be kept constant. To stress, any subset of those layers may be kept constant.

For example, in one specific implementation, a physiologically-based pharmacokinetic model may be used to simulate pharmacokinetic curves for a set of patients with hepatic impairment and a resulting largely decreased clearance. These simulated curves may then be used as retraining data. It is thought that hepatic impairment is unlikely to have an effect on the actual absorption of a given species. With that in mind, when retraining for patients with hepatic impairment, preferably only the curve network is retrained (since the dose effect is unlikely to be affected by the impairment). Specifically, the weights of the dose network may be fixed, and preferably also the LSTM layers of the curve network, since it is acceptable to assume that the LSTM layers can also describe the new curve, and the main changes required are in the nonlinear combination of the parameters defining the curve. As we show later on in this application, retraining the networks by transfer learning in this manner is effective, and leads to good results.

The first and second aspects of the invention relate to computer-implemented methods. It will be appreciated that further aspects of the invention may relate to computer programs and systems for performing the computer-implemented methods of the first aspect of the invention. Specifically, a third aspect of the invention may provide a system for carrying out the computer-implemented method of any one of the first or second aspects of the invention. A fourth aspect of the invention may provide a computer program comprising instructions which, when the program is executed by a computer, cause the computer to carry out the method of either the first or second aspects of the invention. A fifth aspect of the invention may provide a computer-readable medium having stored thereon the computer program of the fourth aspect of the invention.

The preceding disclosure relates to the application of machine learning models of pharmacokinetic models, which relate to how the body processes a given drug. However, it is also envisaged that comparable technical effects could be achieved in respect of pharmacodynamic models (which relate to the effects of a drug on the body), or models taking into consideration both pharmacokinetic and pharmacodynamic effects. Accordingly, in all aspects of the invention, the pharmacokinetic models may be replaced by pharmacodynamic models, or models which take both pharmacokinetics and pharmacodynamics into account.

### BRIEF DESCRIPTION OF THE DRAWINGS

Embodiments and experiments illustrating the principles of the invention will now be discussed with reference to the accompanying figures in which:

FIG. 1 is an example of a system which may be used to train and use a machine learning model to predict a subsequent concentration-time point in a pharmacokinetic curve.

FIG. 2 is a flowchart illustrating the high-level steps involved in using a machine learning model to predict a subsequent concentration-time point in a pharmacokinetic curve.

FIG. 3 is an example of a machine learning model which may be used to predict a subsequent concentration-time point in a pharmacokinetic curve.

FIG. 4 is a series of curves to demonstrate the effects of an LSTM layer on an input sequence.

FIG. 5 demonstrates the time frame over which a dose may be considered to have an effect of the shape of the pharmacokinetic curve.

FIG. 6 illustrates the type of data which may be used to train a dose network.

FIG. 7 illustrates the type of data which may be used to train a curve network.

FIGS. 8A to 8H show results demonstrating the effectiveness of the machine learning model at predicting pharmacokinetic curves in a simulated setting.

FIGS. 9A to 9C show results demonstrating the effectiveness of the machine learning model at predicting pharmacokinetic curves in a real single-dose setting.

FIGS. 10A to 10C show results demonstrating the effectiveness of the machine learning model at predicting pharmacokinetic curves in a real multiple-dose setting.

FIGS. 11A and 11B show results demonstrating the effectiveness of the machine learning model in extrapolating results to new dosage regimens.

FIGS. 12A to 12C show results demonstrating the effectiveness of a retrained version of the machine learning model for hepatically impaired patients.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

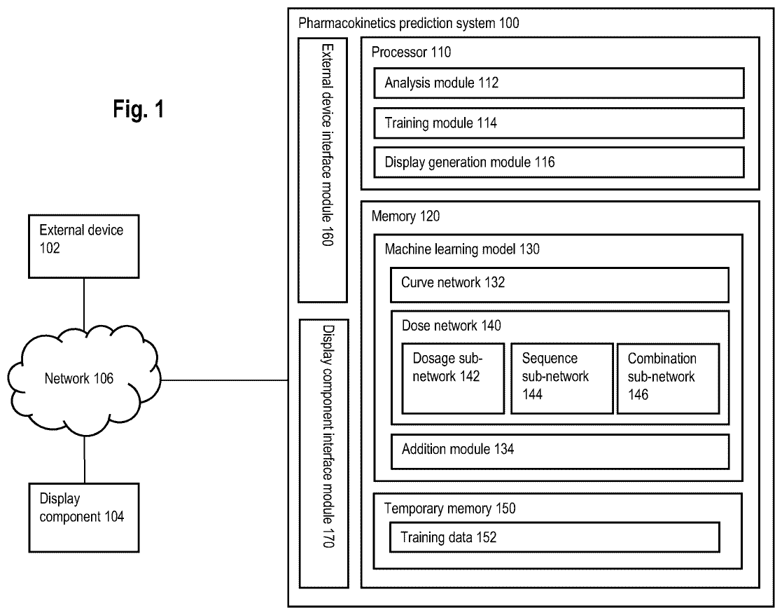

FIG. 1 shows a system 10 which may be used to perform computer-implemented methods of the first and second aspects of the present invention. It should be noted that the system 10 is only an example of the kind of architecture which may be used. Other arrangements of hardware and software are explicitly envisaged. The system comprises pharmacokinetic prediction system 100, external device 102, and display component 104, all of which are connected by network 106. The network may be in the form of a wired or wireless network (e.g. a cellular network or a Wi-Fi network), and may be e.g. a local area network (LAN) or wide area network (WAN). Network 106 may be the internet. In other arrangements, external device 102 and/or display component 104 may be connected directly to the pharmacokinetic prediction system 100. Indeed, the display component 104 may alternatively be part of the pharmacokinetic prediction system 100. In the context of the present application, external device 102 is any device from which input data comprising one or more concentration-time points may be received. For example, external device 102 could be a device configured to measure the concentration of the given species in a user's bloodstream (or other tissue/bodily fluid) and automatically to output the concentration and time point to the pharmacokinetic prediction system 100. Alternatively, external device 102 may be in the form of another device on which the input data may be stored. The external device 102 could, for example, be a computer (e.g. a laptop, desktop, or tablet) or a smartphone, or any other device suitable for storing concentration-time points. In some cases, the external device 102 may be the same physical device as the display component 104, e.g. a computer, or a smartphone, or a piece of bespoke laboratory equipment having a display (specifically equipment configured to measure concentration). The pharmacokinetic prediction system 100, at a high-level is a computer system capable of executing a computer-implemented method, and may be in the form of a regular computer (e.g. a laptop, desktop, or tablet), or could also take the form of a smartphone, or any other device capable of implementing the computer-implemented method of the first and/or second aspects of the present invention. In FIG. 1, the pharmacokinetic prediction system 100 is shown as a single system, however, in some cases, the various components of the pharmacokinetic prediction system 100 may be distributed across several pieces of hardware, which may be connected to each other directly, or via a network (which may or may not be the network 106). It will be noted that the pharmacokinetic prediction system 100 comprises a plurality of functional “modules”. These modules may be in the form of dedicated pieces of hardware which are adapted specifically to perform a prescribed function, or alternatively they may be in the form of functional modules which are implemented in software, e.g. in the form of code and/or instructions which enable a general purpose computer processor to execute the function in question. In some cases, the modules may include a combination of both dedicated hardware and software implementations—as long as such a module is somehow able and adapted to execute the function in question.

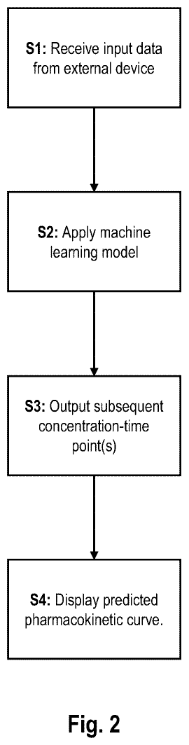

We now discuss the structure of the pharmacokinetic prediction system 100 before discussing its function with reference to FIGS. 2 to 7. The pharmacokinetic prediction system 100 includes processor 110, memory 120, external device interface module 160, and display component interface module 170. The external device interface module 160 acts as an interface between the external device 102 and the pharmacokinetic prediction system 100, and the display component interface module 170 acts as an interface between display component 104 and the pharmacokinetic prediction system 100. The processor 110 includes analysis module 112 for applying the machine learning model 130 to the input data; a training module 114 for training the machine learning model 130; and a display generation module 116 for generating instructions which, when received by the display component 104, cause it to display information indicative of the output of the machine learning model 130. The memory 120 of the pharmacokinetic prediction system 100 includes the machine learning model 130 which itself comprises a curve network 132 and a dose network 140, both of which are preferably in the form of artificial neural networks, whose functions are described elsewhere in this application. The dose network 140 includes three sub-networks: a dosage sub-network 142, a sequence sub-network 144, and a combination sub-network. The machine learning model 132 further comprises an addition module 134 for combining the outputs of the curve network 132 and the dose network 140, preferably by addition, though other combination methods may be used, e.g. weighted addition and the like. Memory 120 of the pharmacokinetic prediction system 100 also includes temporary memory 150, on which the training data 152 may be stored. It is envisaged that after the machine learning model 130 has been trained by the training module 114, the training data 152 may be deleted from the temporary memory 150, as it is no longer required.

We now turn to FIG. 2 which is a high-level flowchart illustrating a computer-implemented method which falls within the scope of the first aspect of the invention, namely a computer-implemented method for predicting a subsequent point on a pharmacokinetic curve. In a first step S1, the input data is received from the external device 102 by the pharmacokinetic prediction system 100, via the external device interface module 160. This step is optional, since the input data in question may already be present on the pharmacokinetic prediction system 100. In order to present a complete view o the invention, in the present example, we consider the case where the method shown in FIG. 2 is used to predict a plurality of concentration-time points, the plurality of concentration-time points encompassing at least two dosing events, and a period in between the dosing events where it is assumed that the administration of a dose no longer has an impact on the concentration-time profile (i.e. the results are governed only by the curve network, as the given species is e.g. metabolized, distributed, or excreted). In order to achieve this, the input data includes three components: a sequence of concentration-time points before the administration of a dose; dosage data including (i) the size of the first dosage and (ii) information indicating the times when subsequent doses will take place as well as size of those doses.

Then, in step S2, the machine learning model 130 is applied to the input data. Specifically, the analysis module 112 of the processor 110 of the pharmacokinetic prediction system 100 may retrieve the machine learning model 130 from the memory 120, and apply it to the input data. The detailed structure of the machine learning model 130 is shown in FIG. 3, and its structure and operation will now be explained in detail. It should be stressed that the example shown in FIG. 3 is illustrative only, and machine learning models having different structures may prove equally effective.

The operation of the machine learning model 130 is generally to take the inputs, apply the various neural networks 132, 140 to that input data, with a view to calculating a subsequent concentration-time point in the pharmacokinetic curve, generally the concentration-time point which would be expected to appear immediately after the most recent point in the sequence of concentration-time points which formed part of the input. Then, an updated input concentration-time sequence, now including the predicted subsequent concentration-time point is input into the machine learning algorithm 132, thereby generating a second subsequent concentration-time point. This process is repeated iteratively in order to obtain a full pharmacokinetic curve.

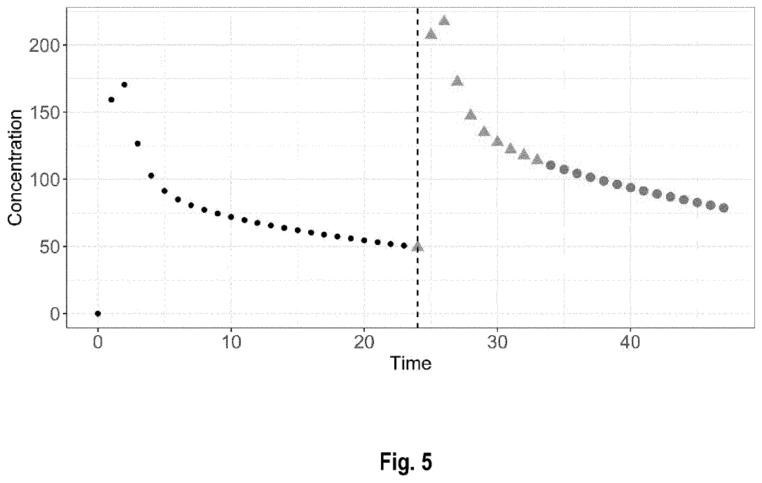

The effects of the actual administration of a dose may not be felt at all times, as we have explained earlier. When the effects of the dose are not felt, it is sufficient to use the curve network 132 alone to determine the subsequent concentration-time point. When the effects are assumed to be felt, then both the curve network 132 and the dose network 140 may be used to predict the subsequent concentration-time point. See e.g. FIG. 5, in which the concentration-time points where the dose effect may be felt are shown as triangles to the right of the dotted line, and the points where no dose effect may be felt are shown as circles, also to the right of the dotted line. The input sequence is shown to the left of the line. All of the input sequence may form the input to the curve network 132, but only e.g. 9 points including the peak may be form the input sequence to the sequence sub-network 144 of the dose network 140.

Accordingly, step S2 (or more generally, the step of applying the machine learning model 130 to the input data) may include a sub-step of determining whether, at the time point for which the concentration-time point is being predicted, the most recent administration of the dose is considered still to have an effect; and if so, using both the curve network 132 and the dose network 140 to predict the subsequent concentration-time point, and if not, using only the curve network 132 to predict the subsequent concentration-time point. The determination may be based on a comparison of the current time point with information determining after how long after the administration of a dose the dose-effect is still felt. This amount of time may be customizable dependent on e.g. the patient in question, or the nature of the given species. As discussed previously, the dose-effect is generally assumed to be felt up to 10 hours after the administration of the dose (though this can vary, see earlier in the application).

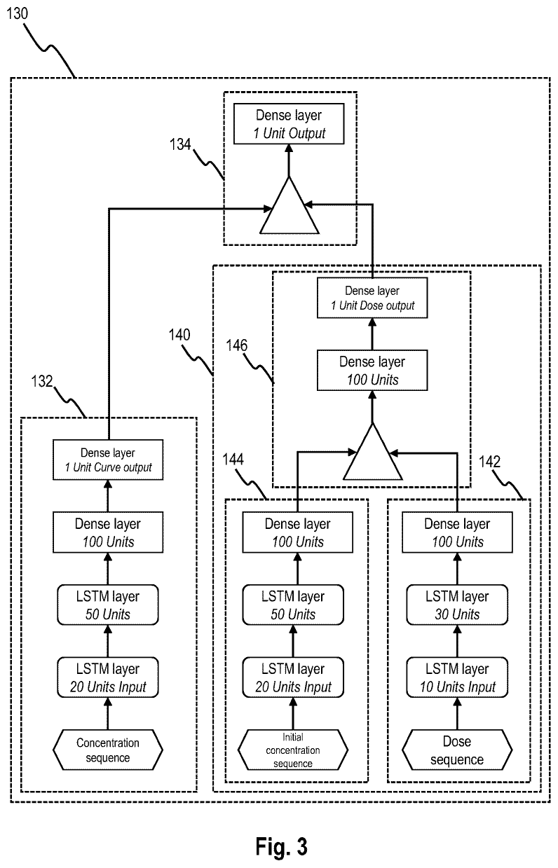

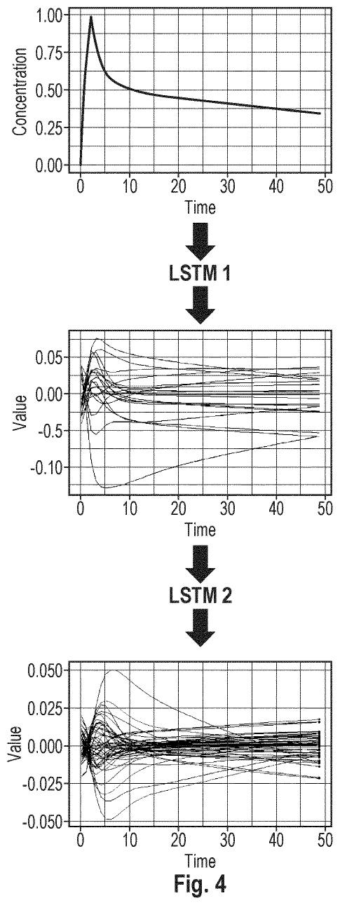

We now consider the curve network 132, and its operation in more detail. FIG. 3 shows the curve network 132. It takes as an input a concentration sequence, which is a sequence of concentration-time points. This data is then passed through two LSTM layers, which in the embodiment depicted in FIG. 3 include, respectively, 20 and 50 units. The effect of these LSTM layers is shown in FIG. 4. The upper pharmacokinetic curve shows the input sequence. The bottom two images show the decomposition of the input sequence into a plurality of curves as a result of the processing by the first, and then the second LSTM layers. What results is a decomposition of the input data into parameters representative of the information contained within. It should be noted that these parameters typically do not correspond to physiologically meaningful parameters. After the processing by the LSTM layers, in the embodiment of FIG. 3, the resulting parameters are passed through two densely connected layers (“Dense layers”) having 100 units and 1 unit respectively. These layers combine the parameters in a nonlinear fashion in order to give rise to a predicted concentration value for the subsequent concentration-time point. It will be noted that the value of the concentration here is not influenced at all by any form of dosage data.

We now consider the dose network 140 and its operation in more detail. The dose network 140 includes a dosage sub-network 142 and a sequence sub-network 142, the results of which are combined using combination sub-network 146. The inputs of the dose network 140 in the embodiment shown comprise an input sequence, which includes a plurality of concentration-time points from a first (measured, or simulated) dosing event. In the specific embodiment, the input sequence includes 9 concentration-time points, covering 9 hours-though this value is optional. The input sequence provides information about the absorption of the dose in the individual subject. The input sequence is processed, within the sequence sub-network 144 using two LSTM layers and a dense layer, as with the curve network 132. The other input to the dose network 140 is dosage data which is provided to the dosage sub-network 142. As discussed previously, the dosage data preferably comprises information about how much drug is administered at which (subsequent) time point, preferably as a multiple of the dose which was administered in the first dosing (which forms part of the input sequence). Because the peak resulting from the first dosage is included in the input sequence, according to the present embodiment, absolute dosage data is not strictly required. The dosage data is processed by two LSTM layers with 10 and 30 units respectively, and a densely connected layer with 100 units. The results from each of the sequence sub-network 144 and the dosage sub-network 142 are then combined in the combination sub-network 146. In this specific embodiment, they may be combined by concatenating the respective outputs of the dosage sub-network 142 and the sequence sub-network 144, and then passing the concatenated outputs through a dense layer with 100 units, and a further dense layer with 1 unit—to generate a combination sub-network 146 output. The concatenation and subsequent processing effectively establish a relationship between the input sequence and the dose sequence, and output a predicted value of the subsequent concentration-time point as a result of the dose effects. It should be noted that this output does not take into account the background decay: this is considered only by the curve network 132.

In a final step, the addition module 134 combines the outputs from the curve network 132 and dose network 140, preferably by simple addition (by the dense layer with 1 unit) in order to generate a final value of the subsequent concentration-time point. This addition ensures that the effects of the background decay in concentration are combined with the effects of the dose.

The process may then be repeated, including the predicted first subsequent concentration-time point in the input sequence of the curve network 132, and optionally the input sequence of the sequence sub-network 144 of the dose network 140. By repeating this process iteratively, a full predicted pharmacokinetic curve may be generated.

In step S3, of FIG. 2, the concentration-time point (s) are output, and then in step S4, the display generation module 116 of the processor 110 of the pharmacokinetic prediction system 100 may generate instructions, which are then sent to the display component 104 via the display component interface module 170. When received at the display component 104, the instructions then cause it to display, e.g. graphically, the output concentration-time point or sequence of points, thereby completing the method of FIG. 2.

To reiterate a point which was made at the end of the “Summary” section, although this specific example pertains to pharmacokinetic models, it is envisaged that the invention would work equally well for predicting subsequent concentration-time points in pharmacodynamic models, or models which take into consideration both pharmacokinetic and pharmacodynamic responses of the body.

### Experimental Results

In order to demonstrate the efficacy of the invention, we now present some experimental results obtained using preferred implementations of the invention. It will be appreciated by the skilled person that, although these results were obtained only for a specific experimental setup, that corresponding technical advantages would also be achieved by alternative specific implementations. As an overview, in the following experiments, it is demonstrated that it is possible to use artificial neural networks of the kind described above to predict concentration-time curves for individual subjects with similar accuracy to ordinary differential equation-based methods. The results show that it is possible to obtain accurate, clinically reliable results with only a limited amount of data. In the experimental workflow, concentration-time profiles are simulated, and the neural networks are trained using the simulated data, and subsequently tested on real clinical data. The ability of the neural network to extrapolate to different dosing schemes is also investigated, as is the transfer of the predictions to a different patient group. The performance metrics used to evaluate the neural networks in this setting include accuracy, generalizability, and computational speed.

### I. Methods

### a) Artificial Neural Network Architecture

In PK models, dosing events manifest in abrupt changes of the system dynamics (e.g. steps in plasma profiles for intravenous dosing or discontinuous first derivatives of plasma profiles for oral dosing). The neural network investigated here, as discussed, aims at predicting of time-concentration profiles at times with and without dosing events.

In the implementation under investigation, the network architecture is structured into two sub-networks: One subnetwork, the curve network, is used to describe the concentration at a following time point when no dose is administered. The other subnetwork, the dose network, is used to describe the concentration increase following a dosing event.

The input to the curve network is a concentration-time profile (sequence) and the output is the next concentration in this sequence. The curve network is composed of two hidden LSTM-layers which decompose a concentration-sequence into parameters representing the shape of the sequence, as illustrated in FIG. 4.

In a subsequent densely connected layer, these parameters are used in a nonlinear combination to predict the concentration at the next time step. The input to the dose network is a sequence of the first 9 concentration-time points from the first dosing as we expect the first concentration peak to lie within this sequence. A second input to the dose network is the dosing-sequence with the doses at each time point relative to the first dose. Both inputs are processed in parallel, each with two hidden LSTM-layers and one densely connected layer, as with the curve network.

The vectors resulting from the densely connected layers were concatenated and processed with three additional densely connected layers. This architecture allows the dose-network to draw a connection between a dose and the concentration increase after a dose in individual patients. The output of the dose-network was added to the output of the curve-network at times of dosing events, as shown in FIG. 3.

### b) Training Data

In order to train the artificial neural network, simulated data from a physiologically based pharmacokinetic (PBPK) model published by Parrott et al¹¹ were used. Single dose simulations were available for 6 mg, 24 mg and 180 mg drug administrations with 400 simulated subjects per dose level.

For multiple doses, data was simulated for 25 subjects with each receiving 10 administrations of 20 mg and a dosing interval of 24 hours. Concentrations were sampled from these simulated concentration-time profiles every hour. In order to streamline the training of the artificial neural network and in accordance with a usual ML workflow, the data were normalized by dividing all concentrations by the maximum concentration following the first drug administration.

### c) Curve Network Training

To train the curve network, both single-dose and multiple-dose data was used. From the single-dose data, 100 subjects were randomly sampled, and the sequence of concentration-time points was split into 80 sequences of random length. Each of the 25 concentration-time sequences with multiple dosing was split into 88 sequences where, for each sequence, the last dosing was at least 10 hours before the end of the sequence, in order to avoid capturing dose effects in the curve network. This is illustrated in FIG. 7. The number of individual sequences generated through this procedure was shown to cover the variability in the training data set. Noise of 10% was added to all sequences, and they were used as input for the curve network. As target-output for the training, we used the subsequent concentration-time point relative to the last concentration-time point in the input sequence. For parameter optimization, an Adam-optimizer¹² was used, which is a standard optimizer for regression functions. As a loss function, a mean-square error was used. After the training of the curve network, the weights of the curve network were fixed, for subsequent training of the dose network, and for making predictions using the curve network.

### d) Dose Network Training

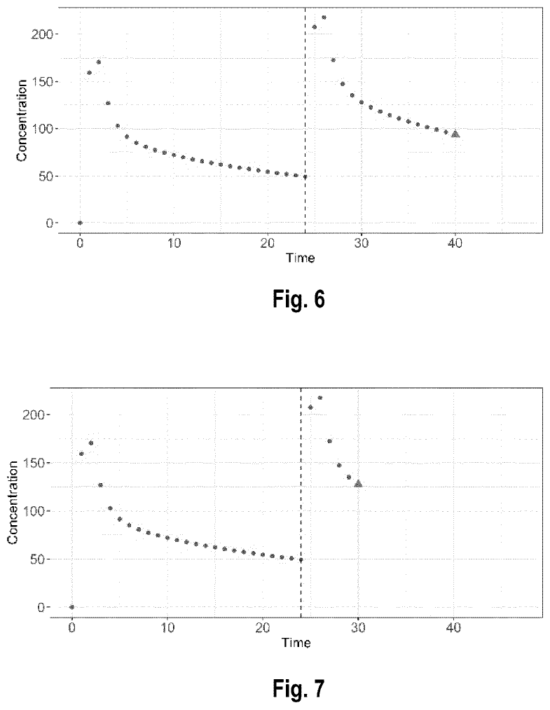

To train the weights of the dose network, the overall network was trained, keeping the weights of the curve network fixed. The 25 simulated concentration-time curves with multiple dosing were split into 71 sequences where the last concentration in the sequence was located in a range up to 10 data points after a previous dosing in order to capture the effect of the new dose, illustrated in FIG. 6. These sequences served as input to the (frozen) curve network. A sequence of the first 9 concentrations of the first dose and a dosing scheme of the corresponding sequence in discrete time steps served as input to the dose network. The 9 concentrations provided information about the absorption of the dose in the individual subject, and the dosing scheme provides information about how much drug was administered at which time step in the sequence. Like with the curve network training, the Adam-optimizer was used, as was a mean-squared error.

### e) Testing on Simulated Data

To assess the sensitivity of the neural network to different training data, 10 neural network variants were trained with 10 different seeds to assemble the random training data.

With each of these 10 neural networks, the complete concentration-time curve was predicted for the same 100 randomly selected subjects from the simulated single dose data set, and for all 25 subjects from the simulated multiple dose data set, in order to test whether the neural networks are able to approximate the shape of the pharmacokinetic concentration-time curves in general. Then, the predicted concentration-time points 1, 10, and 50 time steps ahead for the single dose data, and at the trough and peak predictions of the third, fourth, and fifth dose.

### f) Testing on Real Clinical Data

To investigate the translatability from simulated to observed clinical data, a data set of 53 subjects with single dose administration, and 6 subjects with multiple dose administrations¹³ were used. Measurements from 0 hours to 8 hours were initially selected as the input sequence. Since the concentrations in the single dose study were only measured at 0, 1, 2, 4, and 8 hours, the concentrations at 3, 5, 6, and 7 hours were estimated through logarithmic interpolation. Starting from this input sequence with 9 concentration-time points per subject, the concentration time curves up to 312 hours were predicted though iteratively predicting the next concentration and appending the input sequence with the predicted value. To determine the goodness-of-fit, the measured concentrations were compared to the predicted values at the corresponding time point.

### g) Test for Extrapolation to Different Dose Regimens

A key application for the neural network in clinical pharmacology and precision dosing is the possibility to simulate new dosing regimens. To test whether neural networks are able to extrapolate and make accurate predictions for dose regimens they were not trained on, the physiologically-based pharmacokinetic model was used to simulate additional data. The dose regimens for these simulations included an initial dose of 20 mg followed by 10 mg twice daily after 24 hours or by 40 mg once every second day. The dosing schemes passed to the dose network were adjusted accordingly to predict the whole concentration-time curves based on an initial concentration-time point sequence.

### h) Retraining on New Data

A further challenge in clinical pharmacology is dealing with patient groups with a different pharmacokinetic response, e.g. patients with decreased clearance due to hepatic impairment. As there are no physiological parameters which could be adjusted for these patient groups (which would be the case in “classical” pharmacometrics) a new neural network must be trained separately for a new patient group. In order to minimize the required training data, the neural network on the simulated data for common patients was used for transfer learning. The physiologically-based pharmacokinetic model was used to simulate a data set of patients with hepatic impairment with a largely decreased clearance. The data set was split such that 20 patients were used for the retraining and 50 patients were used to evaluate the retrained neural network. Since hepatic impairment is expected to have no influence on the drug absorption, only the curve network was retrained and investigated. During the retraining, the weights in the LSTM layers were fixed, and only the weights in the densely connected layers were adjusted, because it was assumed that the LSTM layers can also describe the new curve, and the main changes must be done in the nonlinear parameter combination part of the neural network. The retrained neural network was used to predict the whole concentration-time curve for the 50 patients in the test data set. The predicted concentrations at 10 equally distributed time points over the entire prediction time were investigated and compared to the real simulated concentrations.

### II. Results

### a) Neural Networks can Predict Pharmacokinetic Profiles in a Simulated Setting

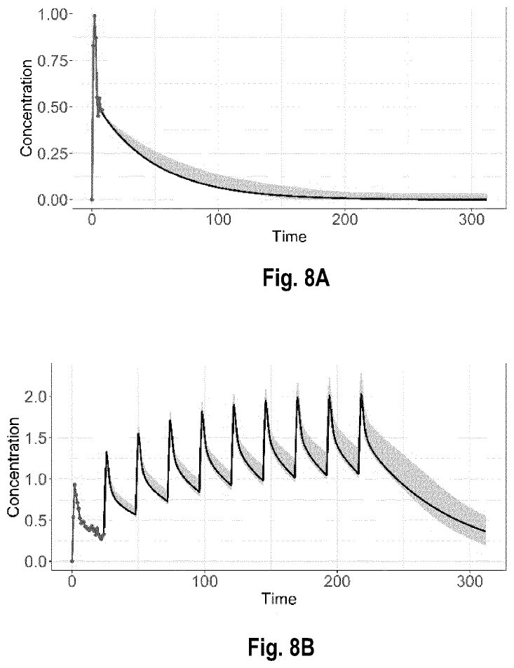

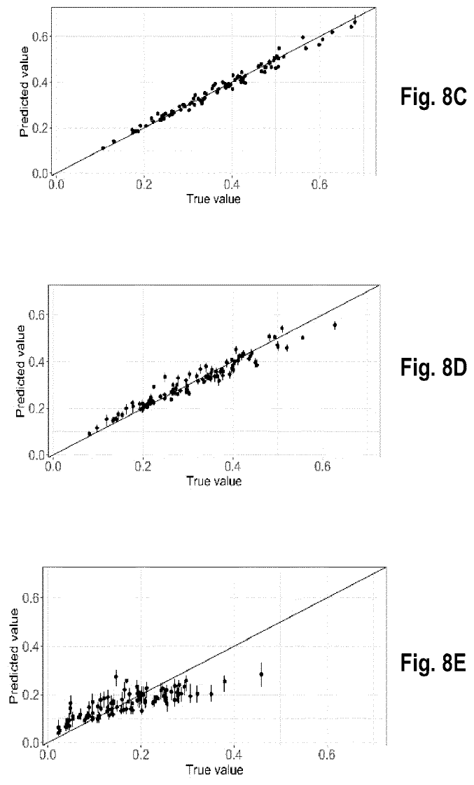

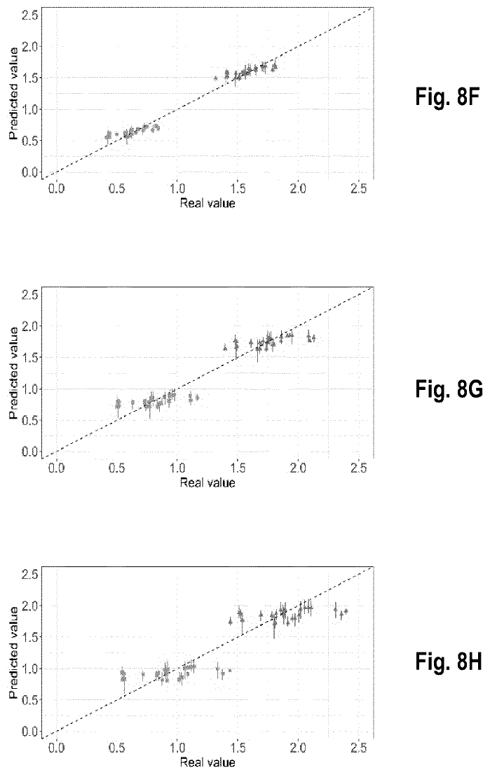

The predicted concentration-time curves of the neural networks for the simulated single and multiple dose data are in agreement with the simulated profiles generated using the underlying ODE model, as demonstrated in FIGS. 8A and 8B, in which an input sequence (shown in the leftmost side of FIGS. 8A and 8B) was given to the 10 trained neural networks. The range of the predictions (the shaded region) covers the underlying simulated concentration-time curve in both examples demonstrating the efficacy of the machine learning model.

In the single dose scenario the exponential decrease is correctly described while in the multiple dose scenario the drug accumulation and the steady state are well-captured by the neural network. Thus, we conclude that in this setting the neural network is capable of producing accurate pharmacokinetic time-concentration profiles.

The single dose predictions for the concentration one step ahead are very close to the simulated values, as shown in e.g. FIG. 8C. With multiple iterations of predicting the next concentration and appending the input to the neural network with the prediction, the residual between the predicted value and the simulated value increases, as shown in FIG. 8D (10-step ahead predictions) and FIG. 8E (50-step ahead predictions).

It is possible to make the same observation for multiple doses looking at the peak and the trough predictions of the third, fourth and fifth dose, as illustrated in FIGS. 8F (third dose), 8G (fourth dose) and 8H (fifth dose). Interestingly, the predicted values for different subjects lay in a rather narrow range while the underlying simulated values differ more from each other.

b) Translation from Simulated to Real Single Dose Data

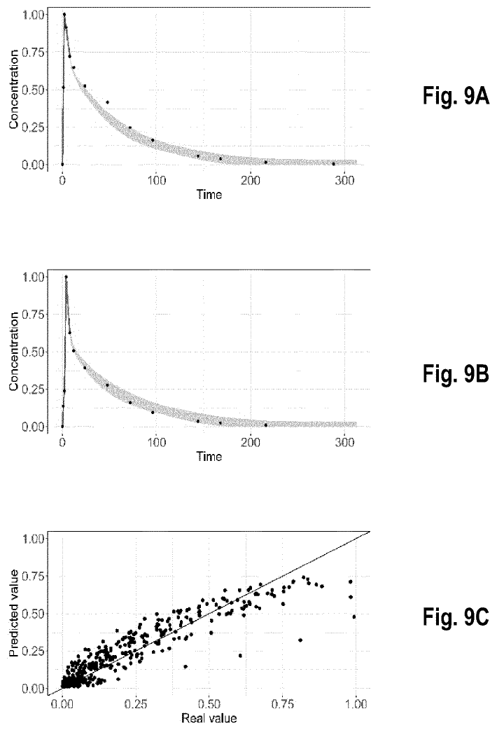

For a similar analysis using observed clinical patient data, the range of predictions of the 10 neural networks covers the real measured concentrations in most cases, as shown in FIGS. 9A to 9C. FIGS. 9A and 9B that with the input sequence shown at the far-left of the graph, the range (shaded) in which predictions of the 10 artificial neural networks lay cover the majority of the real measured concentrations (black dots) for two exemplary subjects with single-dose data. The goodness-of-fit plot in FIG. 9C shows a good correlation between the observed values on the x-axis and the mean predicted values of the y-axis. The neural networks were not only able to make good predictions for on part of the curve but for the entire profile.

The correlation between the mean predictions over all neural networks for each data point and the real measured concentrations is high, with an R² of 0.86. In the goodness-of-fit plot, few outliers can be seen where the neural networks strongly underestimate the concentration. In this case, the absorption was delayed, and the peak concentration was not reached within the input sequence. Since the networks were trained on pharmacokinetic curves with their maximum concentration within the first 9 hours, they were not able to predict this outlier correctly.

c) Translation from Simulated to Real Multiple Dose Data

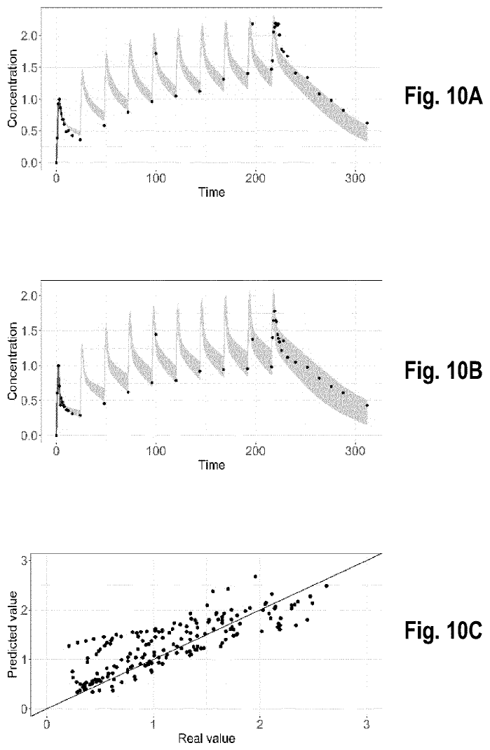

The concentration-time curves were predicted for a multiple-dose schedule using the 10 neural networks, see FIGS. 10A to 10C. FIGS. 10A and 10B that with the input sequence shown at the far-left of the graph, the range (shaded) in which predictions of the 10 artificial neural networks lay cover the majority of the real measured concentrations (black dots) for two exemplary subjects with single-dose data. The goodness-of-fit plot in FIG. 10C shows a good correlation between the observed values on the x-axis and the mean predicted values of the y-axis. With multiple dosing R²=0.75. The real measured concentrations are within the prediction-range of the neural networks for most subjects and the neural networks were able to predict the accumulation and the steady-state well.

### d) Neural Networks can Extrapolate to New Dose Regimens

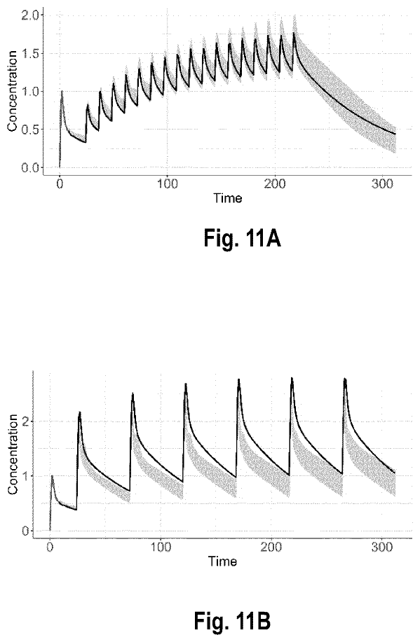

The neural networks made predictions matching the pharmacological understanding for dose regimens which were not included in the initial training data set, see FIGS. 11A and 11B. In these graphs, the similar input sequence (at the left-hand side) with the prediction range of the artificial neural networks (shaded) and the profile simulated by a physiologically-based pharmacokinetic model (dark line) for a dosing with higher dosing frequency but lower doses (FIG. 11A), and for a lower dosing frequency but higher doses (FIG. 11B) show the ability of the neural networks to extrapolate to different dosing regimens.

The difference in the predicted accumulation between the high and the low frequency schedule corresponds to the simulated accumulation. In the high frequency and low dose regimen, we observe a smaller difference between peak and trough concentration while in the low frequency and high dose regimen these differences are larger compared to the original dose schedule.

Also, the biphasic behaviour of the high dose regimen can be observed. There, a slightly larger mismatch between the predicted and the underlying simulated data is observed. This might be because propagation error has a larger influence when the data changes faster as it is in the steep-slope phase.

e) Neural Networks can be Used for New Data without the Need for a Large New Data Set

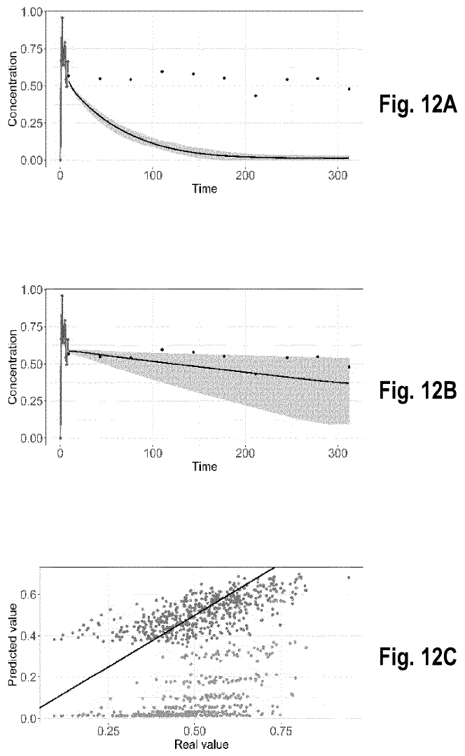

After the transfer learning with a small data set of 20 hepatic impaired patients, the neural networks showed clearly different predictions which were much closer to the concentration time curve of the hepatic impaired patients compared to the original patient population, see FIGS. 12A to 12C. Here, a randomly chosen patient with the input sequence shown at the left-hand side, the range in which the predictions of the ten artificial neural networks lay (shaded) and the mean of the predictions (dark line) are much closer to the real simulated values (dots) after the retraining (FIG. 12B) than compared to before the retraining (FIG. 12A). Also, in the goodness-of-fit in FIG. 12C shown the improvement from without retraining (lighter dots at bottom) compared with the retrained network (darker dots at top).

The mean over the predictions from 10 trained neural networks is close to the true simulated concentrations. However, some of the individual neural networks performed less well and the range of the predictions is larger compared to the predictions for the non-impaired subjects.

### f) Training and Prediction Efficiency

The training of the neural network was performed within one hour on two cores and with 256 GB of RAM. A prediction of a concentration-time profile with 300 prediction steps takes less than 30 seconds. Predictions for multiple subjects can be made in parallel. By comparison, the generation of a suitable model using traditional methods would take several hours to several days-which demonstrates clearly the advantages associated with the use of machine learning methods.

### III. Discussion

The results for simulated data clearly demonstrate that neural networks such as those provided by the present invention are able to make effective, accurate pharmacokinetic predictions in the absence of a predefined pharmacokinetic model. Similarly, the results of the predictions for real data demonstrate the feasibility of training a neural network on simulated pharmacokinetic data, and applying the trained neural network to real clinical data. In this case, a physiologically-based pharmacokinetic model which was previously shown to cover the observed clinical data well was investigated. Therefore, the transition from simulated to actual observed data was feasible without any additional refinements of the neural network.

Neural networks often are assumed to extrapolate poorly to data not included in the training data set. Here, it was seen that the chosen network architecture improves the extrapolations to different dose regimens by providing the explicit information about an increase in the concentration after a dose event, which is connected to the concentration peak following the first dose in an individual subject. With the ability of the neural networks to extrapolate to different dose regimens they qualify for potential applicability in clinical pharmacometrics and precision dosing, in addition to established methods based on ODEs.

The neural networks also showed the ability to translate their predictions from one patient group to another with only a small data set required for the retraining step. This allows one to adapt the predictions quickly to new patient groups. Even though some individual neural networks performed better than others, the mean prediction is good and therefore may be used as a starting point which continuously can be improved through retraining on new incoming data from the new patient group.

The time investment for training a neural network and to make predictions in order of few hours is rather small compared to the conventional pharmacokinetic modelling, especially as this process can be done automatically and does not need constant supervision from the modeler. Furthermore, the application of this method requires only limited pharmacokinetic knowledge while the development of a pharmacokinetic model requires a lot of experience and expertise.

### IV. Conclusion

These experiments show that neural networks, as provided by the present invention, are able to make pharmacokinetic concentration-time predictions for which ODE-based methods are usually used. Their ability to explore dose regimens on which they were not trained showcases their possible application in precision dosing. Also, the possibility to retrain a neural network on small datasets to transfer from one patient group to another shows yet another beneficial property of neural networks in precision dosing. Therefore, neural networks provide an efficient and straightforward supplementary method to conventional pharmacokinetic modelling approaches.

### General Statements

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value.

When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A computer-implemented method of predicting at least one future point on a pharmacokinetic curve for a given species, the computer-implemented method including:
receiving an input comprising data representing a sequence of concentration-time points of a pharmacokinetic curve, each concentration-time point indicative of an amount of the given species in a subject's body at a respective time; and
applying a machine learning model to the input data, the machine learning model configured to generate an output comprising at least one subsequent concentration-time point in the pharmacokinetic curve.

2. The computer-implemented method of claim 1, wherein:
the subsequent concentration-time point of the output is the next concentration-time point after the sequence of concentration-time points forming the input.

3. The computer-implemented method of claim 1, wherein:
the machine learning model comprises:
a curve model comprising a curve network, which is an artificial neural network configured to output one or more subsequent concentration-time points which would be expected in the absence of an administration of a dose of the given species; and
a dose model comprising a dose network, which is an artificial neural network configured to output one or more values indicative of an increase in concentration of the given species after a dose has been administered; and
the input further comprises dosage data including at least one value of a dose to be administered.

4. The computer-implemented method of claim 3, wherein:
the dosage data comprises an absolute dosage value of an initial dosing event, and the times and respective dosage values for at least one subsequent dosing event; and
the dose network is configured to predict the resulting increase in concentration as a result of each of the dosing events described in the dosage data.

5. The computer-implemented method of claim 3, wherein:
the curve network comprises:
at least one long short-term memory, LSTM, layer configured to decompose the sequence of concentration-time points forming the input into parameters representative of the sequence; and
at least one densely connected layer configured to combine the parameters in a nonlinear manner in order to predict the at least one subsequent concentration-time point.

6. The computer-implemented method of claim 3, wherein:
the dose network comprises:
a sequence sub-network configured to receive and process the portion of the input data comprising the sequence of concentration-time points; and
a dosage sub-network configured to receive and process the portion of the input data comprising the dosage data; and
each of the sequence sub-network and the dosage sub-network comprise:
at least one long short-term memory, LSTM, layer configured to decompose a received input into one or more parameters representative of the sequence; and
at least one densely connected layer configured to combine the parameters in a nonlinear manner in order to predict the at least one subsequent concentration-time point.

7. The computer-implemented method of claim 6, wherein:
the dose network further comprises a combination sub-network which is configured to combine the outputs from the dosage sub-network and the sequence sub-network and to output a parameter indicative of the increase in concentration of the given species as a result of the administration of a dosage or plurality of doses, as described by the dosage data.

8. The computer-implemented method of claim 3, further comprising:
adding the output of the dose model to the output of the curve network in order to determine a value for the subsequent concentration-time point.

9. The computer-implemented method of claim 3, wherein:
applying the machine learning model comprises:
(a) applying the curve network to the initial input data to generate a subsequent concentration-time point;
(b) applying the curve network to update input data, the updated input data comprising the initial input data and all subsequently-generated concentration-time points; and
(c) repeating step (b).

10. The computer-implemented method of claim 1, wherein:
the computer-implemented method comprises receiving a plurality of inputs, each input corresponding to a respective dosing regimen and the machine learning model is configured to generate a plurality of outputs, each corresponding to a respective input; and
determining a dosing regimen based on the generated plurality of outputs, each comprising at least one subsequent concentration-time point.

11. The computer-implemented method of claim 10, wherein:
determining a dosing regimen comprises selecting a dosing regimen corresponding to one of the inputs based on its respective output.

12. The computer-implemented method of claim 11, wherein:
determining the dosing regimen comprises determining, for each output, the value of one or more pharmacological parameter; and
selecting the dosing regimen comprises selecting the dosing regimen based on the value of the one or more pharmacological parameter.

13. The computer-implemented method of claim 12, wherein:
the output corresponding to each input comprises a concentration-time curve comprising a plurality of concentration-time points; and
the pharmacological parameter is the area under the respective concentration-time curve (AIC); the proportion of time for which the concentration of the species exceeds a minimum inhibitory concentration (MIC); the AUC/MIC ratio; the maximum concentration value in the concentration-time curve; or the minimum concentration value in the concentration-time curve.

14. The computer-implemented method of claim 12, wherein:
selecting the dosing regimen comprises selecting one or more dosing regimens, the value of the pharmacological parameter calculated for the output corresponding to which is no less than a efficacy threshold; and/or
selecting the dosing regimen comprises selecting one or more dosing regimens, the value of the pharmacological parameter calculated for the output corresponding to which is no more than a predetermined toxicity threshold.

15. The computer-implemented method of claim 12, wherein:
selecting the dosing regimen comprises selecting one or more dosing regimens, the value of the minimum concentration calculated for the output corresponding to which is no less than an efficacy threshold; and
selecting the dosing regiment comprises selecting one or more dosing regimens, the value of the maximum concentration calculated for the output corresponding to which is no more than a toxicity threshold.

16. A computer-implemented method of generating a machine learning model for predicting at least one future point on a pharmacokinetic curve for a given species, the computer-implemented method comprising:
providing a machine learning algorithm; receiving training data, the training data comprising a plurality of pharmacokinetic curves; and
training the machine learning algorithm using the received training data, thereby generating the machine learning model.

17. The computer-implemented method of claim 16, wherein:
the training data includes associated pairs of training data items, the pairs each including an input sequence of concentration-time points, and at least one output concentration-time point.

18. The computer-implemented method of claim 16, wherein:
the machine learning algorithm comprises:
a curve network, which is an artificial neural network configured to output one or more subsequent concentration-time points which would be expected in the absence of an administration of a dose of the given species; and
a dose network, which is an artificial neural network configured to output one or more values indicative of an increase in concentration of the given species after a dose has been administered; and
training the machine learning algorithm comprises:
training the curve network using curve network training data, thereby establishing a plurality of curve network weights;
fixing the curve network weights;
inputting dose network training data comprising at least an input sequence of concentration-time points including a peak concentration-time point immediately after the administration of a dose, and dosage data; and
inputting output data comprising at least one concentration-time point as would be determined by the whole machine learning algorithm.

19. The computer-implemented method of claim 16, further comprising retraining the machine learning algorithm for patients with different pharmacokinetic responses, the pharmacokinetic responses defined by a profile comprising one or more parameters of a physiologically-based pharmacokinetic model which is usable to simulate a patient's pharmacokinetic response, the computer-implemented method further comprising:
generating simulated pharmacokinetic curve training data using the physiologically-based pharmacokinetic model and the parameters in the profile; and
retraining the machine learning algorithm using the simulated pharmacokinetic curve training data.

20. The computer-implemented method of claim 19, wherein:
when retraining the machine learning algorithm, one or more of the following are held constant: one or more LSTM layers of the curve network; one or more densely connected layers of the curve network; one or more LSTM layers of the dose network; and one or more densely connected layers of the dose network.

21. (canceled)
